(19) United States
(12) Patent Application Publication
(10) Pub. No.: US 2003/0191453 A1
(43) Pub. Date: Oct. 9, 2003

(54) Catheter assembly

(76) Inventors: Omar E. Velez, Miramar, FL (US); Richard F. Huyser, Kalamazoo, MI (US); Mark T. Salzberger, Portage, MI (US); Timothy P. Freybler, Battle Creek, MI (US); Richard H. DeVault, Portage, MI (US); Luis A. Diaz, Yabucoa, PR (US); Jason S. Allen, Kalamazoo, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 10/406,761

(22) Filed: Apr. 3, 2003

Related U.S. Application Data
(60) Provisional application No. 60/370,067, filed on Apr. 3, 2002.

(51) Int. Cl. A61M 25/16

(52) U.S. Cl. 604/537; 604/122

(57) ABSTRACT

A catheter assembly for conveying fluid from a fluid source to an anatomical site. A connector portion couples the catheter assembly to the fluid source. The catheter assembly comprises an elongated tube having an open proximal end engaging the connector portion and a closed distal end for inserting into the anatomical site. The elongated tube defines a central lumen and a plurality of apertures in an infusion section thereof to convey the fluid from the central lumen to the anatomical site. An air elimination device is coupled to the elongated tube between the connector portion and the infusion section. Seals are placed between the air elimination device and the elongated tube to prevent air from entering the central lumen of the elongated tube distally of the air elimination device and blocking fluid flow through the plurality of apertures into the anatomical site.

### RELATED APPLICATIONS

This application claims the benefit of U.S. Provisional Patent Application Serial No. 60/370,067, filed Apr. 3, 2002.

### FIELD OF THE INVENTION

The present invention generally relates to a fluid delivery system for delivering fluid to an anatomical site. More specifically, the present invention relates to a catheter assembly of the fluid delivery system utilizing a plurality of apertures for delivering the fluid to the anatomical site.

### BACKGROUND OF THE INVENTION

A typical fluid delivery system comprises a fluid source to hold fluid such as medication. A fluid conduit conveys the fluid from the fluid source to a catheter assembly. The catheter assembly conveys the fluid to an anatomical site of a patient. A connector couples the catheter assembly and fluid conduit.

A typical catheter assembly comprises an elongated tube defining a central lumen and a plurality of apertures at an infusion section of the elongated tube to convey the fluid from the fluid source to the anatomical site. The apertures provide a corridor for the fluid to enter the anatomical site.

An open proximal end of the elongated tube is connected to the connector and a closed distal end of the elongated tube is inserted in the anatomical site. The elongated tube extending from the open proximal end at the connector to the closed distal end at the anatomical site can comprise several sections either integrally formed together, or attached to one another by additional connectors. Either way, the central lumen conveys the fluid from the fluid source to the anatomical site via the plurality of apertures in the infusion section.

Unfortunately, air is easily introduced into the central lumen of the elongated tube due to the connections made throughout the fluid delivery system. For example, the connection of the fluid conduit to the fluid source and the connector may be one source of air infiltration. The connection of the catheter assembly to the connector may be another source. As the air moves toward the closed distal end, the fluid is obstructed from exiting out of the plurality of apertures in the infusion section of the elongated tube. Ultimately, due to the small diameter of the apertures, the air becomes permanently entrapped in the central lumen at the infusion section. The air either settles at the closed distal end of the elongated tube or gets trapped along the central lumen in the proximity of the apertures and permanently blocks a portion of the plurality of apertures in the infusion section.

Current pain management pump systems used with catheter assemblies utilize devices to remove air from the pump system upstream of a restrictor orifice. However, the devices used in such systems are upstream of one or more connections to the fluid conduit, connector, or catheter assembly. Thus, air infiltrates the connections and moves through the central lumen to the infusion section of the tube as previously described. Alternatively, to remove the air, a user primes the elongated tube using a syringe by injecting the fluid at a high flow rate and pressure into the open proximal end of the elongated tube while the elongated tube is disconnected from the connector. This action is intended to drive or push the air out through the plurality of apertures. Many problems are associated with this activity. For instance, even if the priming of the elongated tube removes a large percentage of the air, the air rushes in through the open proximal end when the user removes the syringe and makes the connection with the fluid conduit. In addition, assuming the priming removes most of the air, air can still be infiltrated at connection points such as between the connector and the elongated tube. Hence, even if the elongated tube is primed, not all of the air can be removed in current systems.

Accordingly, there is a need in the art to prevent the air from transcending through the central lumen of the elongated tube and restricting the uniform flow of fluid out of the plurality of apertures at the infusion section of the elongated tube.

### BRIEF SUMMARY OF THE INVENTION

The present invention provides a catheter assembly for conveying fluid from a fluid source to an anatomical site. A fluid connection mechanism provides fluid communication between the catheter assembly and the fluid source. The catheter assembly comprises an elongated tube having an open proximal end and a closed distal end. The open proximal end is coupled to the fluid connection mechanism to receive the fluid. The closed distal end enters the anatomical site to deliver the fluid. The elongated tube defines a central lumen and a plurality of apertures between the open proximal and closed distal ends. The fluid from the fluid source enters the elongated tube through the central lumen at the open proximal end and exits the elongated tube through the plurality of apertures near the closed distal end. An air elimination device is coupled to the elongated tube between the open proximal end and the plurality of apertures to remove trapped air from the central lumen to ensure that the air does not reach the closed distal end or block the fluid from exiting through the plurality of aperture to the anatomical site.

The catheter assembly of the present invention overcomes the disadvantages of the prior art by removing air from the fluid in the fluid delivery system thereby eliminating obstruction of the plurality of apertures in the elongated tube and providing uniform fluid delivery. Uniform fluid delivery, especially when delivering medication for pain relief is essential in providing adequate pain relief to a patient.

### BRIEF DESCRIPTION OF THE SEVERAL VIEWS OF THE DRAWINGS

Advantages of the present invention will be readily appreciated as the same becomes better understood by reference to the following detailed description when considered in connection with the accompanying drawings wherein:

FIG. 1 is an elevational view of one embodiment of the fluid delivery system of the present invention;

FIG. 1A is an elevational view of another embodiment of the fluid delivery system;

FIG. 2 is cross-sectional view of a catheter assembly of the fluid delivery system taken along line 2-2 of FIG. 1 to illustrate a reinforcing coil of the catheter assembly;

FIG. 3 is a fragmentary perspective view of one embodiment of an elongated tube of the catheter assembly of the present invention illustrating an infusion section of the elongated tube;

FIG. 3A is a fragmentary perspective view of another embodiment of the elongated tube of the catheter assembly of the present invention illustrating the infusion section of the elongated tube defining a vent hole;

FIG. 3B is a fragmentary perspective view of another embodiment of the elongated tube of the catheter assembly of the present invention illustrating the infusion section of the elongated tube defining a vent hole in a tip of the elongated tube;

FIG. 4 is an elevational view of an alternative embodiment of the fluid delivery system of the present invention;

FIG. 5 is a fragmentary perspective view of an elongated tube of a catheter assembly of the alternative embodiment illustrating a support in an infusion section of the elongated tube;

FIG. 6 is a fragmentary perspective view of the elongated tube of the catheter assembly of the alternative embodiment illustrating the support in a continuous section of the elongated tube;

FIG. 7 is a cross-sectional view taken along the line 7-7 of FIG. 4 to illustrate a guide system of the present invention;

FIG. 8 is a cross-sectional view taken along the line 8-8 of FIG. 4 to illustrate the guide system of the present invention; and

FIG. 9 is a perspective view of a support tip of the guide system of the present invention.

### DETAILED DESCRIPTION OF THE INVENTION

Referring to the Figures, wherein like numerals indicate like or corresponding parts throughout the several views, a fluid delivery system for delivering fluid to an anatomical site 10 of a patient 11 is generally shown at 12. It is to be appreciated that the fluid can be medication, irrigation fluid such as a saline solution, and the like. The fluid delivery system could be used to supply the fluid to the anatomical site 10, to irrigate the anatomical site 10, or other related functions.

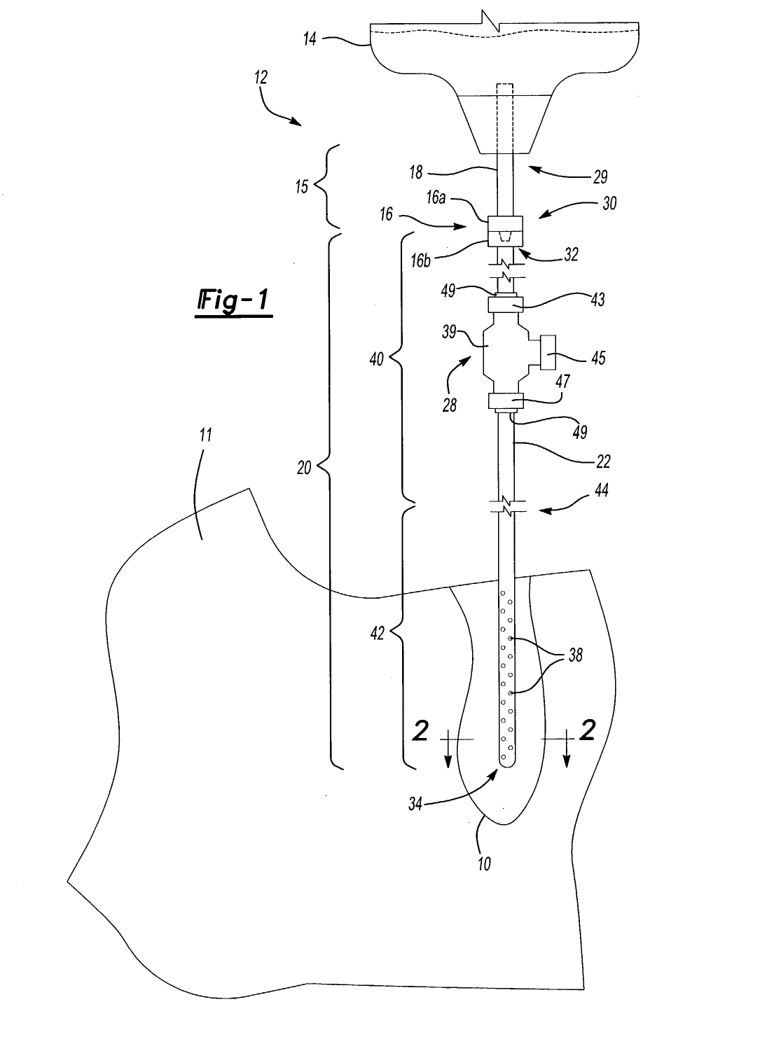

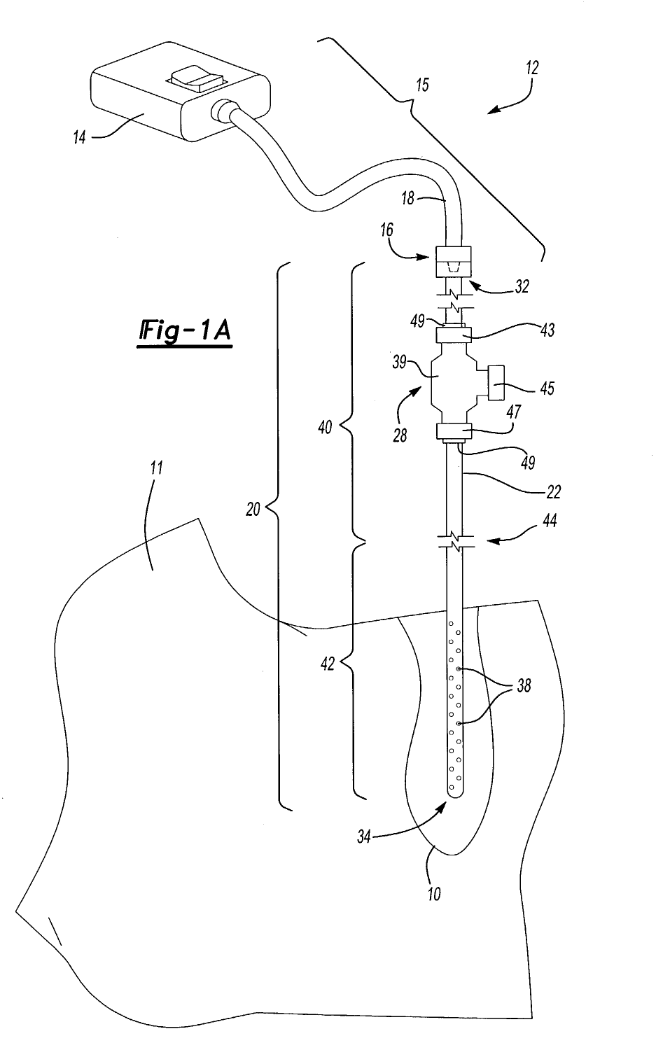

Referring to FIGS. 1 and 1A, the fluid delivery system 12 includes a fluid source 14 to supply the fluid to be delivered to the anatomical site 10. The fluid source 14 can include any mechanism or manner to store and/or deliver the fluid to the anatomical site 10 of the patient. Such mechanisms may include, but are not limited to, pain medication pumps, intravenous bags, syringes to inject fluid, and the like. FIG. 1 illustrates the use of an intravenous bag as the fluid source 14 and FIG. 1A illustrates the use of a pain medication pump as the fluid source 14.

A fluid conduit 18 is in fluid communication with the fluid source 14 and conveys the fluid from the fluid source 14 to a catheter assembly 20. The fluid conduit 18 includes a proximal end 29 coupled to the fluid source 14 and a distal end 30 coupled to the catheter assembly 20. For purposes of this embodiment, the fluid conduit 18 is generally illustrated as one possible configuration of a tube set 15 for the fluid delivery system 12. Those skilled in the art will appreciate that any tube set, i.e., a series of tubes and connectors, may be used to couple the fluid source 14 to the catheter assembly 20. For instance, the fluid conduit 18 may include multiple tubes, a stopcock to control a flow rate of the fluid, a converter adapted to couple different diameter tubes, and the like.

The fluid conduit 18 may be made from any number of materials, as recognized by those skilled in the art, including, but not limited to, any appropriate flexible polymer, sterilizable and biocompatible in the case of medical applications. Some typical materials include silicone, polyamide, polyether block amide, polyethylene, urethane, polyurethane, fluorinated ethylene propylene, PTFE, other fluoropolymers, and the like.

A fluid connection mechanism 16, such as a connector 16 couples the fluid conduit 18, i.e., tube set 15, with the catheter assembly 20. It should be appreciated that the catheter assembly 20 may directly engage the fluid source 14 by other well-known fluid connection mechanisms including, but not limited to, bonding, welding, or by being integrally formed therewith. Hence, eliminating the need for a fluid conduit 18 and connector 16.

The connector 16 comprises a first connector portion 16A engaging the fluid conduit 18 at the distal end 30 and a second connector portion 16B engaging the catheter assembly 20. The connector portions 16A, 16B interlock to form the connector 16 thereby operatively interconnecting the fluid source 14 with the catheter assembly 20. The connector portions 16A, 16B are preferably Luer-lock quick connect fittings. Of course, the connector portions 16A, 16B may be threaded fittings, snap fittings, or any type of fittings used by those skilled in the art to interlock the fluid conduit 18 and the catheter assembly 20.

The connector 16 may be made from any number of materials, as recognized by those skilled in the art, including, but not limited to, any appropriate polymer, sterilizable and biocompatible in the case of medical applications. Some typical materials include silicone, polyamide, polyether block amide, polyethylene, urethane, polyurethane, fluorinated ethylene propylene, PTFE, other fluoropolymers, and the like.

The catheter assembly 20 includes an elongated tube 22 in fluid communication with the fluid source 14 to convey the fluid from the fluid source 14 to the anatomical site 10. The elongated tube 22 extends between an open proximal end 32 and a closed distal end 34. The open proximal end 32 engages the second connector portion 16B. Preferably, the open proximal end 32 is scaled to the second connector portion 16B to provide a fluid-tight seal therewith. For instance, the second connector portion 16B may be integrally formed with the elongated tube 22 at the open proximal end 32 or the second connector portion 16B may be welded to the elongated tube 22 at the open proximal end 32 by way of a solvent weld, ultrasonic weld, thermal weld, adhesive, or the like.

The elongated tube 22 defines a central lumen 24 and a plurality of apertures 38 therein for conveying the fluid from the fluid source 14 to the anatomical site 10. A continuous section 40 and an infusion section 42 define the elongated tube 22. The continuous section 40 extends from the open proximal end 32 to an intermediate point 44 and the infusion section 42 extends from the intermediate point 44 to the closed distal end 34. It is to be appreciated that the differentiation between the continuous and infusion sections 40,42 is for descriptive purposes and does not indicate that the elongated tube 22 is discontinued or interrupted at the intermediate point 44. The continuous section 40 is free of apertures, while the plurality of apertures 38 are defined in the infusion section 42 to deliver the fluid to the anatomical site 10.

Referring to FIG. 3, each of the plurality of apertures 38 preferably have a diameter 46 between 0.0001 inches to 0.025 inches to convey the fluid from the central lumen 24 to the anatomical site 10. It is to be understood, however, that the present invention can be used with any sized aperture 38. The apertures 38 preferably assume a circular shape, but may take on a variety of shapes including elliptical, rectangular, triangular, or a combination of two or more different shapes. The apertures 38 may also be in the form of slots. See FIG. 3B. The plurality of apertures 38 are preferably laser-drilled into the elongated tube 22, but alternative methods may also be used to form the apertures 38.

Referring to alternative embodiments of the infusion section 42 of the elongated tube 22, shown in FIGS. 3A and 3B, the elongated tube 22 may further define a vent hole 37 distal to the plurality of apertures 38. The vent hole 37 is used to remove residual air that may otherwise may be trapped in the central lumen 24 of the elongated tube 22 between a tip 41 of the elongated tube 22 at the distal end 34 and the plurality of apertures 38. When the catheter assembly 20 of FIG. 1 is being primed, using methods well known to those skilled in the art, air is forced out through the plurality of apertures 38 and the vent hole 37.

Referring to the embodiment of FIG. 3A, the vent hole 37 is defined between the plurality of apertures 38 and the tip 41. In this embodiment, the plurality of apertures 38 are axially and equally spaced from one another along said elongated tube by a first length L1. The vent hole 37 is axially spaced from said plurality of apertures by a second length L2. The second length L2 is shorter than the first length L1. This configuration allows the plurality of apertures 38 to maintain uniform fluid delivery along the infusion section 42, while still providing a vent for air that may be trapped near the tip 41. Of course, in this embodiment, a small space is still available for air to be trapped, but this space is reduced considerably by the use of the vent hole 37. The vent hole 37 in the embodiment of FIG. 3A assumes the same size and shape as the apertures 38.

Referring to the embodiment of FIG. 3B, the vent hole 37 is centrally positioned in the tip 41 and has a diameter smaller than the plurality of apertures 38. The vent holes of FIGS. 3A and 3B are also laser-drilled into the elongated tube 22.

The apertures 38 illustrated in FIGS. 3-3B are radially disposed from a central axis of the elongated tube 22 by one hundred twenty degrees, as illustrated. Hence, in these embodiments, three apertures 38 lie in each of a plurality of axially spaced planes (not illustrated) that are perpendicular to the central axis. The axially spaced planes are spaced by the length L1.

The elongated tube 22 can be made from any number of materials including, but not limited to, any appropriate flexible polymer, sterilizable and biocompatible in the case of medical applications. Some typical materials include silicone, polyamide, polyether block amide, polyethylene, urethane, polyurethane, fluorinated ethylene propylene, PTFE, other fluoropolymers, and the like.

Referring back to FIG. 1, an air elimination device 28, such as an air filter, is coupled to the elongated tube 22 between the open proximal end 32 and the plurality of apertures 38 to remove air from the central lumen 24. This ensures that air does not reach the infusion section 42 and block the fluid from exiting through the plurality of apertures 38 to the anatomical site 10. As previously discussed, air trapped in the infusion section 42 can obstruct one or more of the plurality of apertures 38 resulting in non-uniform fluid delivery. As can be appreciated by those skilled in the art, uniform fluid delivery is important in many medical procedures. In the present instance, while air embolism is a concern, the size of the plurality of apertures 38 is usually too small to permit the air to exit the central lumen 24 through the plurality of apertures 38 into the anatomical site 10, at least under normal operating pressures. Usually, excess pressure, such as by priming the catheter assembly 20, is required to force any air from the central lumen 24 through the plurality of apertures 38.

The air elimination device 28 is in fluid communication with the central lumen 24 of the catheter assembly 20. In essence, the air elimination device 28 separates any air or gas from the fluid and allows the air-free fluid to continue through the elongated tube 22, while expelling the air to the atmosphere. Preferably, the air elimination device 28 includes a liquid-permeable, gas-impermeable filter (not shown) and a liquid-impermeable, gas-permeable filter (not shown) positioned within a filter housing 39 to filter or remove any air from the fluid to facilitate uniform fluid delivery via the plurality of apertures 38.

The filter housing 39 is coupled to the elongated tube 22 and defines an inlet port 43, a vent port 45, and an outlet port 47. The filter housing 39 may be one piece or multiple pieces bonded together using well-known methods. The fluid, with air or gas, enters the filter housing 39 via the inlet port 43, and the air or gas is separated from the fluid when the fluid moves through the liquid-permeable, gas-impermeable filter to the outlet port 47. The air or gas that is prevented from exiting the filter housing then passes through the liquid-impermeable, gas-permeable filter and exits to the atmosphere through the vent port 45. The filtered fluid continues from the outlet port 47 to the infusion section 42. Hence, in this instance, the liquid-permeable, gas-impermeable filter is a barrier to the outlet port 47 and the liquid-impermeable, air-permeable filter is a barrier to the vent port 45.

Seals 49 are placed between the elongated tube 22 and the filter housing 39 at both the inlet port 43 and the outlet port 47. These seals 49 ensure that air cannot enter the catheter assembly 20 by way of connection between the elongated tube 22 and the air elimination device 28. Hence, a closed fluid path is defined along said elongated tube 22 between the open proximal end 32 and the plurality of apertures 38. The seals 49 may be solvent welds, ultrasonic welds, thermal welds, adhesives, and the like, or the seals 49 may result from the filter housing 39 being integrally molded with the elongated tube 22. In this embodiment, there are no other connections distal to the air elimination device 28. This further reduces the chance for air to enter the central lumen 24 at the infusion section 42 and block fluid flow through the plurality of apertures 38.

The air elimination device 28 can be made from any number of materials. For instance, the filter housing 39 may be made from any appropriate polymer or metal, sterilizable and biocompatible in the case of medical applications. Some typical materials include acrylic, ABS, polycarbonate, polypropylene, PVC, acetal, polyethylent, and the like. The filters described above may be a combination of a hydrophilic membrane and a hydrophobic membrane, or a composite membrane with combined characteristics. Materials for the filters may include, but are not limited to, PTFE, polyethersulfone, nylon, acrylic, polysulfone, and polypropylene. Factors such as sterilization, strength, flexibility, and weight may be taken into consideration in determining the materials to be used. The material used for the air elimination device 28 is not intended to limit the present invention.

Suitable air elimination devices 28 for use in the fluid delivery system 12 of the present invention are shown in U.S. Pat. No. 4,571,244 to Knighton and U.S. Pat. No. 6,503,225 to Kirsch et al., both herein incorporated by reference.

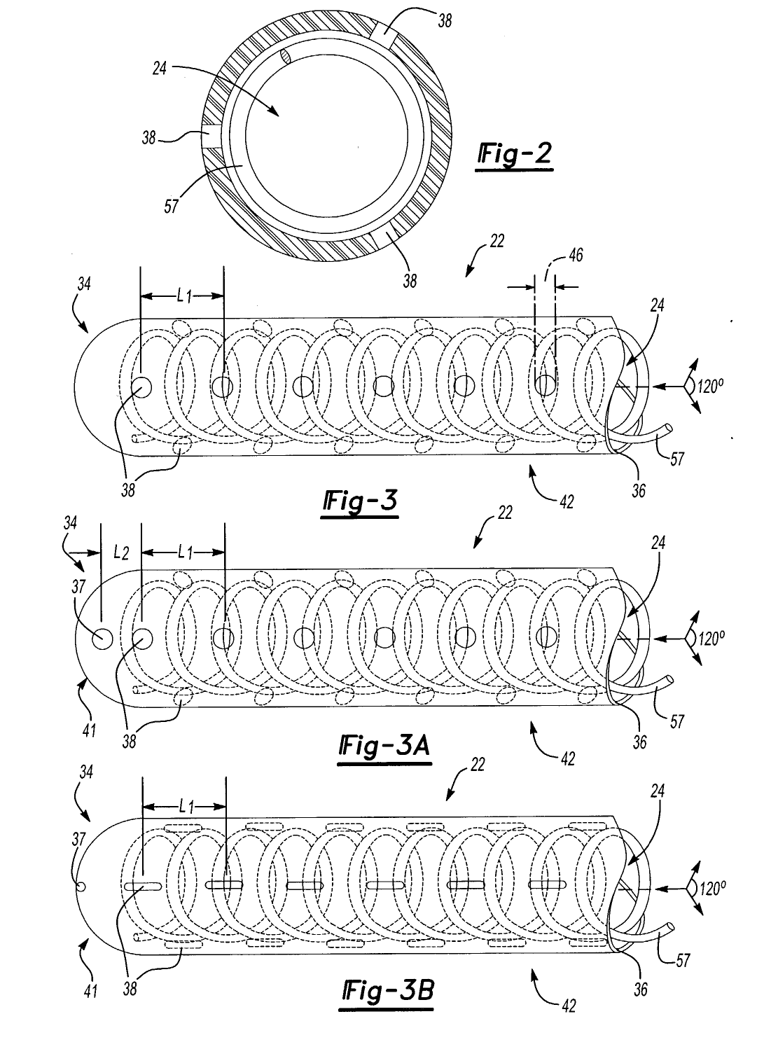

A reinforcing coil 57 is positioned in the central lumen 24 between the air elimination device 28 and the tip 41. See FIGS. 2 and 3-3B. The reinforcing coil 57 freely floats within the central lumen 24 to reinforce the elongated tube 22 to prevent kinking along the infusion section 42. The reinforcing coil 57 has spaced convolutions in an unstretched state. The fluid moves through and along the reinforcing coil 57.

The reinforcing coil 57 may be made from any appropriate metal, sterilizable, biocompatible, and rust-resistant in the case of medical applications, such as stainless steel, and the like.

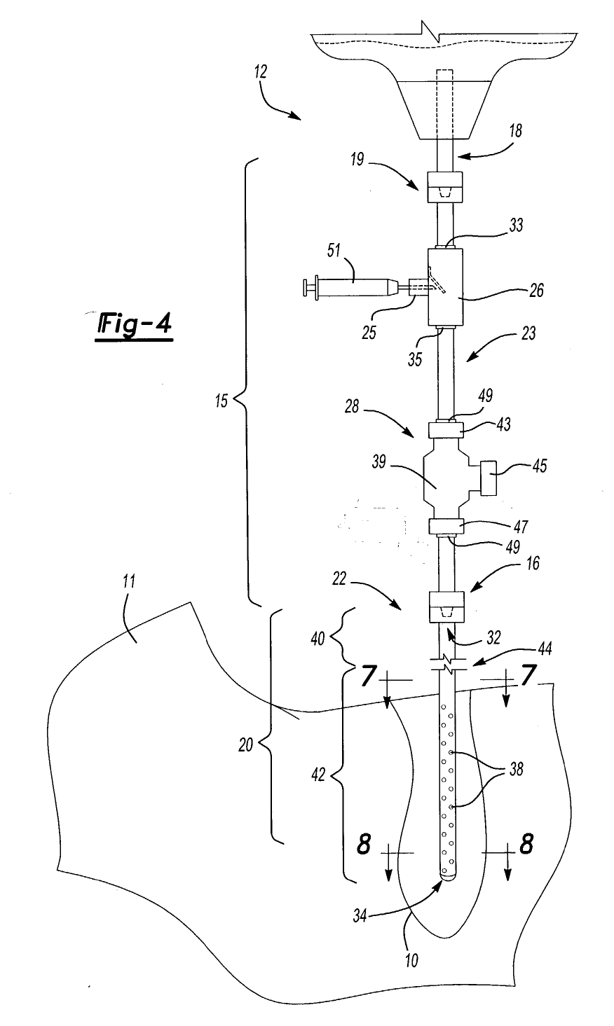

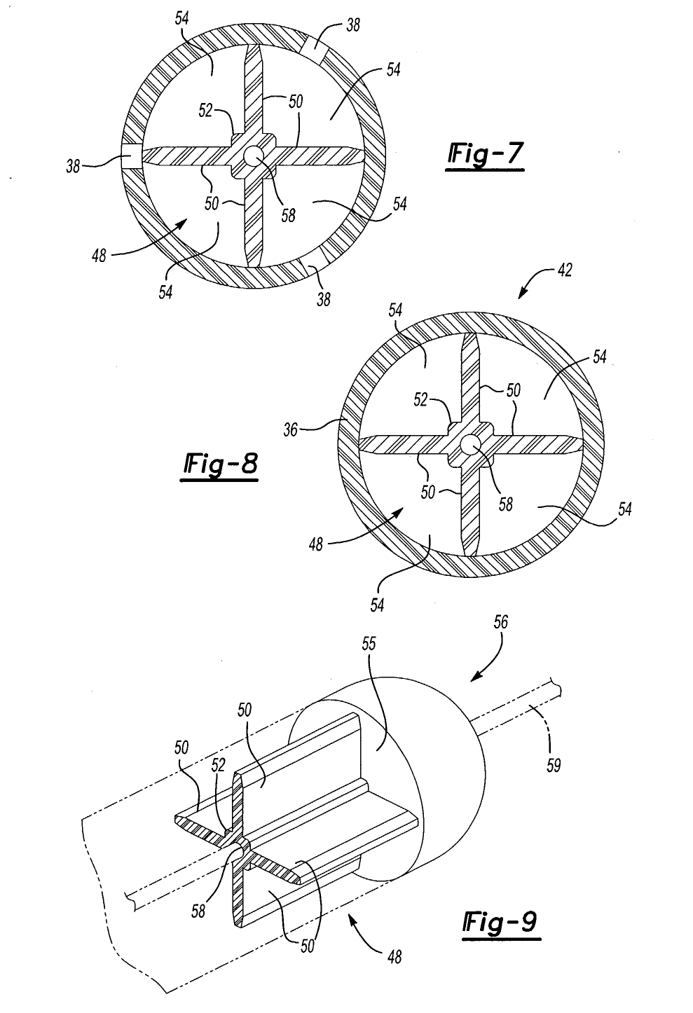

Additional alternative embodiments of the fluid delivery system 12 will now be described. In one alternative embodiment, shown in FIGS. 4-9, the fluid delivery system 12 is the same as the previous embodiments set forth above, with three notable exceptions. First, the tube set 15 of this alternative embodiment includes additional tubes and connectors. In this embodiment, a second connector 19, similar to the connector 16, couples the fluid conduit 18 to a second fluid conduit 23, and the catheter assembly 16 is coupled to the second fluid conduit 23. Second, the air elimination device 28 is coupled to the second fluid conduit 23 of the tube set 15. Third, a guide system is illustrated for inserting the catheter assembly 20 into the anatomical site 10.

A housing 26 defining a priming port 25 is coupled to the tube set 15. The priming port 25 provides a one-way valve, or check valve, to allow a user to prime the catheter assembly 20 prior to use, i.e., the priming port 25 is in fluid communication with the central lumen 24. The housing 26 is a T-shaped or Y-shaped fitting that permits the flow of fluid therethrough to the central lumen 24. The housing defines an inlet port 33 for receiving the fluid from the second fluid conduit 23 and an outlet port 35 for conveying the fluid back to the second fluid conduit 23. Priming the catheter assembly 20 removes the air from the catheter assembly 20 to ensure uniform fluid delivery. The user primes the catheter assembly 20 by injecting the fluid at a high pressure into the priming port 25, thus driving the air out through the plurality of apertures 38. Generally, a syringe 51 is used to inject the fluid into the priming port 25, as shown in FIG. 4.

The housing 26 may be made from any appropriate polymer or metal, sterilizable and biocompatible in the case of medical applications. Some typical materials include acrylic, ABS, polycarbonate, polypropylene, PVC, acetal, polyethylent, and the like. The one-way valve or check valve may be made from any appropriate material, sterilizable and biocompatible in the case of medical applications. Some typical materials include, silicone, PVC, nitrile, latex, neoprene, urethane, polyurethane, fluoropolymers, and the like. Furthermore, the priming port 25 may assume any configuration that effectively allows the user to prime the catheter assembly 20.

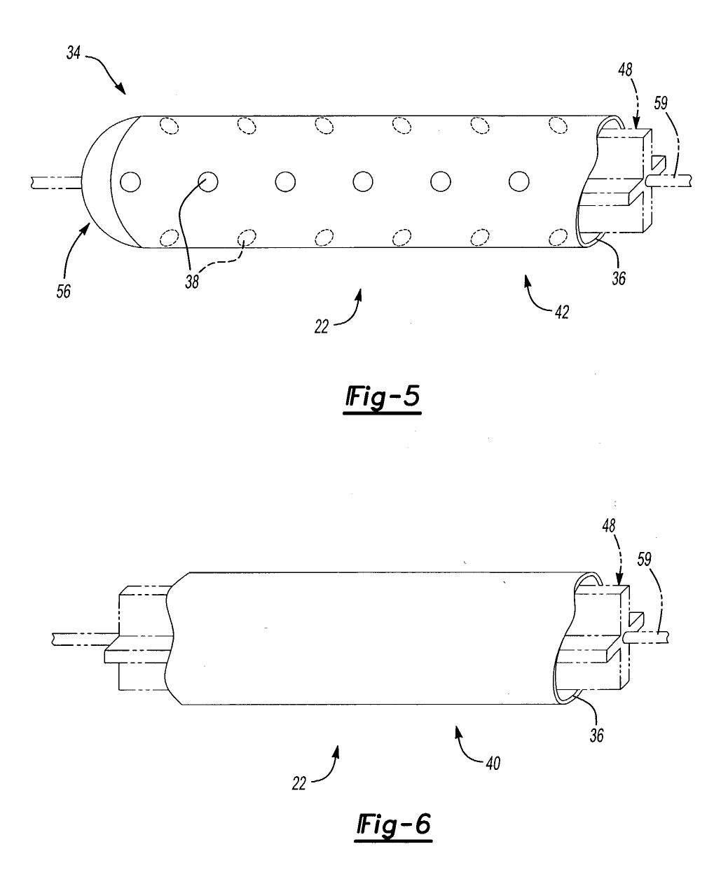

Referring to FIGS. 5-9, the guide system for inserting the catheter assembly 20 into the anatomical site 10 is generally illustrated. The guide system includes a support 48 having a plurality of ribs 50 radially extending from a base 52 of the support 48. The support 48 extends longitudinally through the central lumen 24 of the elongated tube 22 such that the plurality of ribs 50 radially extend from the base 52 toward an inner surface 36 of the elongated tube 22. The support 48 partially performs the function of the reinforcing coil 57 in the previously described embodiment.

The support 48 essentially divides the central lumen 24 into a plurality of lumens 54 to convey the fluid to the anatomical site 10. A support tip 56 is formed at a distal end of the support 48. The support tip 56 is integrally formed with the support 48. The elongated tube 22 is sealed about the support 48 and support tip 56 such that the fluid that flows through the plurality of lumens 54 can only be discharged through the plurality of apertures 38. Furthermore, the distal end 34 of the elongated tube 22 in this embodiment is open and abuts a ledge 55 of the support tip 56. The elongated tube 22 is sealed to the support tip 56 at the ledge 55, as shown by the hidden lines in FIG. 9. The base 52 of the support 48 defines a guide wire channel 58 to receive the guide wire 59.

The support 48 and support tip 56 can be formed from a variety of materials, giving due consideration to the goals of flexibility, weight, strength, and the like. Suitable materials include nylon, polyamide, Teflon, and the like.

Well-known methods and tube set configurations can be employed to insert the catheter assembly 20 into the anatomical site 10 using the guide system. Such methods and configurations will not be described in detail. Any suitable configuration should provide for sliding the catheter assembly 20, via the guide wire channel 58, over the guide wire 59 into the anatomical site, removing the guide wire 59 from the guide wire channel 58 once the catheter assembly 20 is in place in the anatomical site, and then occluding fluid flow through the guide wire channel 58 into the anatomical site. Once the catheter assembly has been primed and positioned into the anatomical site 10, the air elimination device 28 functions to remove any additional air that may be introduced in the fluid proximally to the air elimination device 28 during use.

Of course, the embodiment illustrated in FIG. 4 may be practiced without the guide system. Instead, the catheter assembly 20 illustrated in FIGS. 1, 1A, 2, and 3A-3B may be used in the embodiment of the fluid delivery system 12 of FIG. 4. In such an instance, one of the air elimination devices 28, either on the tube set 15, or the elongated tube 22, would be eliminated. Similarly, the housing 26 with priming port 25 may be coupled to the fluid conduit 18 of the embodiment illustrated in FIGS. 1, 1 A, 2, and 3-3B. Either way, the combination of the housing 26 with the priming port 25 and the air elimination device 28 distal to the priming port 25 is used to remove unwanted air.

It is to be understood that the catheter assembly 20 is not limited to a single tube unit, that is, a single lumen. For instance, a multiple tube catheter assembly may be used that includes two or more tubes from several fluid sources converging into a single catheter assembly. Similarly, a single fluid source may be connected onto a one-inlet/multiple outlet adapter so that a catheter assembly may be connected to each outlet to provide fluid delivery to multiple anatomical sites. The embodiments of the fluid delivery system 12 described herein are not limited in application. For example, each of the embodiments may be used in high-pressure and high-flow rate systems, low-pressure and low-flow rate systems, or any combination thereof.

Obviously, many modifications and variations of the present invention are possible in light of the above teachings. The invention may be practiced otherwise than as specifically described within the scope of the appended claims.

What is claimed is:

1. A catheter assembly for conveying fluid from a fluid source to an anatomical site comprising:
a fluid connection mechanism for providing fluid communication between said catheter assembly and the fluid source;
an elongated tube having an open proximal end coupled to said fluid connection mechanism and extending to a closed distal end and defining a central lumen and a plurality of apertures between said open proximal and closed distal ends whereby the fluid from the fluid source enters said central lumen at said open proximal end and exits through said plurality of apertures near said closed distal end; and
an air elimination device coupled to said elongated tube between said open proximal end and said plurality of apertures for removing air from said central lumen to ensure that trapped air does not reach said closed distal end or block the fluid from exiting through said plurality of apertures to the anatomical site.

2. A catheter assembly as set forth in claim 1 further including at least one seal between said air elimination device and said elongated tube for defining a closed fluid path along said elongated tube between said open proximal end and said plurality of apertures.

3. A catheter assembly as set forth in claim 2 wherein said seal comprises at least one of a solvent weld, ultrasonic weld, thermal weld, and adhesive.

4. A catheter assembly as set forth in claim 1 further including a reinforcing coil having spaced convolutions in an unstretched state and disposed in said central lumen of said elongated tube between said air elimination device and said closed distal end.

5. A catheter assembly as set forth in claim 2 wherein said elongated tube defines a continuous section and an infusion section whereby said continuous section is free of apertures to further define the closed fluid path and said infusion section includes said plurality of apertures to deliver the fluid to the anatomical site.

6. A catheter assembly as set forth in claim 5 wherein said plurality of apertures are laser-drilled in said elongated tube.

7. A catheter assembly as set forth in claim 6 wherein said plurality of apertures comprise at least two different shapes to facilitate uniform fluid delivery.

8. A catheter assembly as set forth in claim 5 wherein said elongated tube further defines a vent hole near said closed distal end wherein each of said plurality of apertures are axially and equally spaced from one another along said elongated tube by a first length and said vent hole is axially spaced from said plurality of apertures by a second length smaller than said first length thereby reducing space in said closed distal end for air to become entrapped.

9. A catheter assembly as set forth in claim 5 wherein said elongated tube further defines a vent hole centrally located in said closed distal end wherein said vent hole has a first diameter and each of said plurality of apertures have a second diameter greater than said first diameter.

10. A catheter assembly as set forth in claim 1 wherein each of said plurality of apertures have a diameter between 0.0001 inches and 0.025 inches.

11. A fluid delivery system for uniformly delivering fluid to an anatomical site comprising:
a fluid source;
an elongated tube in fluid communication with said fluid source and defining a central lumen and a plurality of apertures therein for uniformly conveying the fluid from said fluid source to the anatomical site;
an air elimination device positioned along said elongated tube and sealed to said elongated tube for defining a closed fluid path between said air elimination device and said plurality of apertures to prevent air from entering said elongated tube distally of said air elimination device; and
a reinforcing coil having spaced convolutions in an unstretched state and disposed in said central lumen of said elongated tube for reinforcing said elongated tube to prevent kinking and improve fluid delivery.

12. A fluid delivery system as set forth in claim 11 wherein said plurality of apertures are laser drilled into said elongated tube and comprise at least two different shapes to facilitate fluid delivery.

13. A fluid delivery system as set forth in claim 12 wherein each of said plurality of apertures have a diameter between 0.0001 inches and 0.025 inches.

14. A fluid delivery system as set forth in claim 11 further including a fluid conduit having a proximal end coupled to said fluid source and extending to a distal end.

15. A fluid delivery system as set forth in claim 14 further including a connector having a first connector portion engaging said distal end of said fluid conduit and a second connector portion engaging said first connector portion.

16. A fluid delivery system as set forth in claim 15 wherein said elongated tube includes an open proximal end engaging said second connector portion and extends to a closed distal end.

17. A fluid delivery system for uniformly delivering fluid to an anatomical site comprising:
a fluid source;
a fluid conduit having a proximal end in fluid communication with said fluid source and extending to a distal end;
a connector comprising a first connector portion engaging said fluid conduit and a second connector portion engaging said first connector portion;
an elongated tube having an open proximal end coupled to said second connector portion and extending to a distal end wherein said elongated tube defines a central lumen and a plurality of apertures between said open proximal and distal ends for uniformly conveying the fluid from said fluid source to the anatomical site;
a housing coupled to said fluid conduit and having a priming port in fluid communication with said central lumen for priming said elongated tube to force air out from said central lumen prior to use; and
an air elimination device distal to said housing and coupled to one of said fluid conduit and said elongated tube whereby said air elimination device removes air during use of said fluid delivery system and said priming port removes air prior to use of said fluid delivery system.

18. A fluid delivery system as set forth in claim 17 wherein said elongated tube defines a continuous section defining a closed fluid path and an infusion section distally extending from said continuous section and further defining said plurality of apertures.

19. A fluid delivery system as set forth in claim 17 further including a reinforcing coil having spaced convolutions in an unstretched state and disposed in said central lumen of said elongated tube between said air elimination device and said distal end.

20. A catheter assembly as set forth in claim 19 wherein said elongated tube further defines a vent hole near said distal end wherein each of said plurality of apertures are axially and equally spaced from one another along said elongated tube by a first length and said vent hole is axially spaced from said plurality of apertures by a second length smaller than said first length thereby reducing space in said distal end for air to become entrapped.

21. A catheter assembly as set forth in claim 19 wherein said elongated tube further defines a vent hole centrally located in said distal end wherein said vent hole has a first diameter and each of said plurality of apertures have a second diameter greater than said first diameter.

22. A fluid delivery system for uniformly delivering fluid to an anatomical site comprising:
a fluid source;
an elongated tube having an open proximal end in fluid communication with said fluid source and extending to a distal end wherein said elongated tube defines a central lumen and a plurality of apertures between said open proximal and distal ends for uniformly conveying the fluid from said fluid source to the anatomical site;
a housing having a priming port in fluid communication with said central lumen for priming said elongated tube to force air out from said central lumen prior to use; and
an air elimination device distal to said housing and in fluid communication with said central lumen whereby said air elimination device removes air during use of said fluid delivery system and said priming port removes air prior to use of said fluid delivery system.

23. A catheter assembly as set forth in claim 22 further including a reinforcing coil having spaced convolutions in an unstretched state and disposed in said central lumen of said elongated tube between said air elimination device and said distal end.

24. A catheter assembly as set forth in claim 23 wherein said plurality of apertures are laser-drilled in said elongated tube.

25. A catheter assembly as set forth in claim 24 wherein said elongated tube further defines a vent hole near said distal end wherein each of said plurality of apertures are axially and equally spaced from one another along said elongated tube by a first length and said vent hole is axially spaced from said plurality of apertures by a second length smaller than said first length thereby reducing space in said distal end for air to become entrapped.

26. A catheter assembly as set forth in claim 24 wherein said elongated tube further defines a vent hole centrally located in said distal end wherein said vent hole has a first diameter and each of said plurality of apertures have a second diameter greater than said first diameter.

27. A catheter assembly as set forth in claim 22 wherein each of said plurality of apertures have a diameter between 0.0001 inches and 0.025 inches.
